(19) United States
(12) Patent Application Publication
(10) Pub. No.: US 2025/0073286 A1
(43) Pub. Date: Mar. 6, 2025

(54) A PROBIOTIC COMPOSITION FOR IMPROVING WHEY PROTEIN PROTEOLYSIS, AUGMENTING AMINO ACID PRODUCTION, AND IMPROVING LACTOSE DEGRADATION ABILITY

(71) Applicant: LACTOMASON CO., LTD., Jinju-si, Gyeongsangnam-do (KR)

(72) Inventors: Minn SOHN, Jinju-si (KR); So Lim SHIN, Anyang-si (KR); Woo Hyun JUNG, Seoul (KR); Young Jin LEE, Seoul (KR)

(21) Appl. No.: 18/391,729

(22) Filed: Dec. 21, 2023

Related U.S. Application Data
(63) Continuation of application No. PCT/KR2023/015414, filed on Oct. 6, 2023.

(30) Foreign Application Priority Data
Sep. 1, 2023 (KR) 10-2023-0116275

(51) Int. Cl. A61K 35/747; A61P 1/14

(52) U.S. Cl. CPC A61K 35/747; A61P 1/14

(57) ABSTRACT

Mixed strains composition capable of improving whey protein proteolysis and lactose intolerance, and more particularly mixed strains composition of Limosilactobacillus reuteri LM1071 (Accession Number: KCCM12650P) and Lactobacillus gasseri LM1065 (Accession Number: KCCM13018P), which is capable of degrading whey protein to branched chain amino acid (BCAA), arginine, and phenylalanine and improving lactose intolerance is proposed.

A composition including mixed strains of Limosilactobacillus reuteri LM1071 (Accession Number: KCCM12650P) and Lactobacillus gasseri LM1065 (Accession Number: KCCM13018P) has excellent protein proteolysis and thus can produce BCAA (valine, leucine and isoleucine), arginine, and phenylalanine, which are helpful in forming muscles and suppressing muscle loss, by degrading whey proteins, and has excellent β-galactosidase activity and thus can hydrolyze lactose. Therefore, the mixed strains composition can be used for foods, health functional foods and infant formulas for improving indigestion, diarrhea, abdominal distension, etc. caused by protein intake.

### TECHNICAL FIELD

The present disclosure relates to mixed strains composition capable of improving whey protein proteolysis and lactose intolerance, and more particularly to mixed strains composition of Limosilactobacillus reuteri LM1071 (Accession Number: KCCM12650P) and Lactobacillus gasseri LM1065 (Accession Number: KCCM13018P), which is capable of degrading whey protein to branched chain amino acid (BCAA), arginine, and phenylalanine and improving lactose intolerance.

### BACKGROUND

Muscle loss associated with aging and an increase in obese population caused by westernized diet and less physical activity are important from a public health perspective. Protein intake is helpful in maintaining muscle while suppressing muscle loss associated with aging, and intake of sufficient amounts of proteins by all age groups is known to reduce the risk of sarcopenia and diseases caused by obesity.

Representative dietary sources of proteins can be classified into animal proteins such as milk, eggs, beef, pork, chicken, and fish and vegetable proteins such as soybeans and grains. Milk protein among the protein sources has an excellent composition of essential amino acids, and whey protein concentrate (WPC) produced by concentrating 80% or more of whey, which is a liquid by-product separated from milk during a cheese or casein production stage, is highly bioavailable and quickly absorbed. Thus, WPC has been widely consumed as a protein supplement.

WPC has been reported to various biological activities such as in vivo formation of muscles, prevention of gout, regulation of blood cholesterol levels, improvement of immunity, promotion of bone growth and prevention of obesity, and is also known to be high in essential amino acids. In particular, branched chain amino acids (BCAA) such as valine, leucine and isoleucine are essential for maintenance of metabolic balance of normal proteins and synthesis of muscles, and can reduce muscle damage caused by exercise and promote synthesis of muscle proteins.

A dietary protein such as WPC is a high molecular substance and thus needs to be degraded to a molecular substance after intake. However, in the body, peptide chains of the protein are degraded by various digestive enzymes such as pepsin in the stomach, and trypsin and chymotrypsin in the small intestine and finally absorbed by the intestinal villi in the form of amino acids. In this process, enzymes secreted by gut microbes present in the digestive system also affect degradation and absorption of proteins. Since the gut microbes can secrete enzymes which cannot be biosynthesized by humans, the types and contents of amino acids produced by degradation of the dietary protein may greatly vary depending on the composition of the gut microbes and intake of probiotics. Arginine, one of amino acids produced by degradation of a protein, is known to be helpful in increasing blood nitric oxide levels, eliminating lactic acid and ammonia in the muscle produced during exercise, increasing blood flow and thus promoting glucose uptake, and repairing muscle fibers by means of contractility and activation of satellite cells surrounding the muscle. Also, phenylalanine, one of essential amino acids, is known to be helpful in increasing muscle strength by stimulating synthesis of muscle proteins and increasing exercise performance ability. Therefore, the types of amino acids produced by degradation of a protein may affect muscle synthesis, exercise performance ability and recovery after exercise.

Meanwhile, protein intake may bring about various physiological effects such as formation of muscle and improvement of immunity. However, since a protein is a high molecular substance, it can cause indigestion depending on personal digestive health conditions and a gas generated during degradation of the protein may cause heavy stomach, abdominal distension, fart, foul-smelling stools, etc.

Since WPC, which has been widely consumed as a protein supplement, is a high-protein food composed of 70 to 85% proteins, the intake of WPC may cause heavy stomach and abdominal distension. Also, WPC contains about 5% lactose. When lactose intolerance is caused by deficiency of lactase, a digestive enzyme that hydrolyzes lactose, the lactose is not hydrolyzed in the digestive system after intake of WPC, which may result in gastrointestinal disorders such as stomachache, diarrhea and vomiting.

Although lactose intolerance occurs when the small intestine has a disease or is damaged or excised, most cases occur due to an innate deficiency of lactase. Lactose intolerance is a common disease and the number of patients with lactose intolerance accounts for about 67% of the entire worldwide population. However, the incidence of lactose intolerance significantly varies between races. It is known that about 10% of the European population and about 75% of the Korean population have lactose intolerance. Also, the incidence of lactose intolerance increases as people grow up. It is known that about 25% of the Korean school-age population and about 75% of the Korean adult population have lactose intolerance. Further, the incidence of lactose intolerance differs between sexes. It is known that 80% of the Korean adult male population and 73% of the Korean adult female population have lactose intolerance.

Therefore, probiotics capable of promoting digestion of WPC, which has been widely used as a dietary protein, in the digestive system, degrading the ingested WPC to BCAA and arginine and hydrolyzing lactose is expected to have high industrial utility.

Conventional examples of probiotic compositions having protein proteolysis may include a food composition and health functional food comprising Lacticaseibacillus rhamnosus IDCC 3201 having protein proteolysis (Korean Patent No. 10-2423025). However, there is still a need for development and studies on compositions having better effects.

Accordingly, the present inventors have made efforts to develop a probiotic composition with improved whey protein proteolysis, and as a result, have completed the present disclosure by developing mixed strains composition of Limosilactobacillus reuteri LM1071 (Accession Number: KCCM12650P) and Lactobacillus gasseri LM1065 (Accession Number: KCCM13018P), which is capable of degrading whey protein and improving lactose intolerance.

### DISCLOSURE OF THE INVENTION

### Problems to be Solved by the Invention

The present disclosure is conceived to provide mixed strains composition capable of improving whey protein proteolysis and lactose intolerance.

However, the problems to be solved by this disclosure are not limited to those mentioned above, and other problems not mentioned will be clearly understood by a person with ordinary skill in the art from the following description.

### Means for Solving the Problems

A first aspect of the present disclosure provides mixed strains capable of improving whey protein proteolysis and lactose intolerance.

A second aspect of the present disclosure provides a food composition comprising the mixed strains of the first aspect or fragments, a culture, and extracts of the strain as active ingredients and capable of improving whey protein proteolysis and lactose intolerance.

A third aspect of the present disclosure provides a health functional food composition comprising the mixed strains of the first aspect or fragments, a culture, and extracts of the strain as active ingredients and capable of improving whey protein proteolysis and lactose intolerance.

A fourth aspect of the present disclosure provides an infant formula composition comprising the mixed strains of the first aspect or fragments, a culture, and extracts of the strain as active ingredients and capable of improving protein proteolysis and lactose intolerance.

A fifth aspect of the present disclosure provides a comprising administering to a mammalian subject effective amount of Limosilactobacillus reuteri LM1071 (Accession Number: KCCM12650P) and Lactobacillus gasseri LM1065 (Accession Number: KCCM13018P).

### Effects of the Invention

A composition comprising mixed strains of Limosilactobacillus reuteri LM1071 (Accession Number: KCCM12650P) and Lactobacillus gasseri LM1065 (Accession Number: KCCM13018P) has excellent protein proteolysis and thus can produce BCAA (valine, leucine, and isoleucine), arginine, and phenylalanine, which are helpful in forming muscles and suppressing muscle loss, by degrading whey proteins, and has excellent β-galactosidase activity and thus can hydrolyze lactose. Therefore, the mixed strains composition can be used for foods, health functional foods and infant formulas for improving indigestion, diarrhea, abdominal distension, etc. caused by protein intake.

### BRIEF DESCRIPTION OF THE DRAWINGS

FIG. 1 shows a result of comparative measurement on whey protein proteolysis of mixed strains depending on a mixing ratio of Limosilactobacillus reuteri LM1071 and Lactobacillus gasseri LM1065.

FIG. 2 shows a result of comparative measurement on the amounts of valine, leucine and isoleucine and the total amount thereof depending on a mixing ratio of strains in a test to identify branched chain amino acid production potential by mixed strains of Limosilactobacillus reuteri LM1071 and Lactobacillus gasseri LM1065.

FIG. 3 shows a result of comparative measurement on arginine depending on a mixing ratio of strains in a test to identify arginine production potential by mixed strains of Limosilactobacillus reuteri LM1071 and Lactobacillus gasseri LM1065.

FIG. 4 shows a result of comparative measurement on phenylalanine depending on a mixing ratio of strains in a test to identify phenylalanine production potential by mixed strains of Limosilactobacillus reuteri LM1071 and Lactobacillus gasseri LM1065.

### BEST MODE FOR CARRYING OUT THE INVENTION

Hereafter, examples of the present disclosure will be described in detail with reference to the accompanying drawings so that the present disclosure may be readily implemented by a person with ordinary skill in the art. However, it is to be noted that the present disclosure is not limited to the examples but can be embodied in various other ways. In the drawings, parts irrelevant to the description are omitted for the simplicity of explanation, and like reference numerals denote like parts through the whole document.

Throughout this document, the term “connected to” may be used to designate a connection or coupling of one element to another element and includes both an element being “directly connected to” another element and an element being “electronically connected to” another element via another element.

Further, through the whole document, the term “comprises or includes” and/or “comprising or including” used in the document means that one or more other components, steps, operation and/or existence or addition of elements are not excluded in addition to the described components, steps, operation and/or elements unless context dictates otherwise.

Through the whole document, the term “about or approximately” or “substantially” is intended to have meanings close to numerical values or ranges specified with an allowable error and intended to prevent accurate or absolute numerical values disclosed for understanding of the present disclosure from being illegally or unfairly used by any unconscionable third party. Through the whole document, the term “step of” does not mean “step for”.

Through the whole document, the term “combination(s) of” included in Markush type description means mixture or combination of one or more components, steps, operations and/or elements selected from a group consisting of components, steps, operation and/or elements described in Markush type and thereby means that the disclosure includes one or more components, steps, operations and/or elements selected from the Markush group.

Through the whole document, a phrase in the form “A and/or B” means “A or B, or A and B”.

Hereinafter, embodiments and examples of the present disclosure will be described in detail. However, the present disclosure may not be limited to the following embodiments and examples.

A first aspect of the present disclosure provides a composition comprising mixed strains and capable of improving whey protein proteolysis and lactose intolerance.

In an embodiment of the present disclosure, the mixed strains may have a mixing ratio of Limosilactobacillus reuteri LM1071 and Lactobacillus gasseri LM1065 of 2:8 to 4:6, preferably 3:7, but is not limited thereto. In an embodiment of the present disclosure, it was confirmed that the proteolysis significantly varies depending on a mixing ratio of the strains. In particular, it was confirmed that the mixed strains of Limosilactobacillus reuteri LM1071 and Lactobacillus gasseri LM1065 shows the highest whey protein proteolysis at a mixing ratio of 3:7.

In an embodiment of the present disclosure, the mixed strains may have branched chain amino acid production potential, but is not limited thereto.

In an embodiment of the present disclosure, the mixed strains having branched chain amino acid production potential may have a mixing ratio of Limosilactobacillus reuteri LM1071 and Lactobacillus gasseri LM1065 of 9:1 to 6:4, preferably 9:1, but is not limited thereto. In an embodiment of the present disclosure, it was confirmed that the potential of degrading WPC to branched chain amino acids significantly varies depending on a mixing ratio of the strains. In particular, it was confirmed that the mixed strains of Limosilactobacillus reuteri and Lactobacillus gasseri shows the highest whey protein proteolysis at a mixing ratio of 9:1.

In an embodiment of the present disclosure, the mixed strains may have α-galactosidase activity and β-galactosidase activity, but is not limited thereto.

A second aspect of the present disclosure provides a food composition comprising the mixed strains of the first aspect or fragments, a culture, and extracts of the strain as active ingredients and capable of improving whey protein proteolysis and lactose intolerance. The features described above in respect of the first aspect of the present disclosure may equally apply to the food composition according to the second aspect of the present disclosure.

In an embodiment of the present disclosure, the food composition may contain the mixed strains of the present disclosure or a culture, fragments, and extracts of the strain as active ingredients, but is not limited thereto.

Through the whole document, the term “food” may include meats, sausages, breads, chocolates, candies, snacks, cookies, pizza, ramens, other noodles, gums, dairy products including ice cream, soups, beverages, teas, drinks, alcohol drinks, vitamin complexes, health functional foods and health foods, and may include all foods in the accepted meaning.

The food of the present disclosure can be manufactured by conventional methods used in the art, and can be manufactured by adding conventional raw materials and ingredients used in the art. Further, a formulation of the food is not limited as long as the formulation is accepted as a food. The food composition of the present disclosure may be prepared in a variety of formulations. Since the food is used as raw materials, unlike general drugs, the food composition is free from side effects which may occur when a drug is taken for a long time and may have excellent portability. Therefore, the food of the present disclosure may be taken as a supplement for enhancing the effects of improving protein proteolysis and lactose intolerance.

Since the food composition of the present disclosure can be routinely ingested, the food composition is expected to show a high efficacy on the improvement of lactose intolerance and thus can be very usefully applied.

The food composition may further contain a physiologically acceptable carrier. The kind of the carrier is not particularly limited. Any carrier may be used as long as it is commonly used in the art.

Further, the food composition may further contain additional ingredients that are commonly used in food compositions so as to improve smell, taste, visuality, etc. For example, the food composition may contain vitamins A, C, D, E, B1, B2, B6, B12, niacin, biotin, folate, pantothenic acid, etc. Furthermore, the food composition may also contain minerals such as zinc (Zn), iron (Fc), calcium (Ca), chromium (Cr), magnesium (Mg), manganese (Mn), copper (Cu), chromium (Cr), etc. Moreover, the food composition may also contain amino acids such as lysine, tryptophane, cysteine, valine, etc.

Further, the food composition may also contain food additives, such as preservatives (potassium sorbate, sodium benzoate, salicylic acid, sodium dehydroacetate, etc.), disinfectants (bleaching powder, higher bleaching powder, sodium hypochlorite, etc.), antioxidants (butylhydroxyanisole (BHA), butylhydroxytoluene (BHT), etc.), colorants (tar color, etc.), color-developing agents (sodium nitrite, etc.), bleaching agents (sodium sulfite), seasonings (monosodium glutamate (MSG), etc.), sweeteners (dulcin, cyclemate, saccharin, sodium, etc.), flavors (vanillin, lactones, etc.), swelling agents (alum, potassium D-bitartrate, etc.), fortifiers, emulsifiers, thickeners (adhesive pastes), film-forming agents, gum base agents, antifoaming agents, solvents, improvers, etc. The additives may be selected and used in an appropriate amount depending on the type of food.

The mixed strains of Limosilactobacillus reuteri LM1071 and Lactobacillus gasseri LM1065 of the present disclosure may be added as it is or may be used in conjunction with other foods or food ingredients, and may be appropriately used according to a conventional method. The mixing amount of active ingredients may be appropriately determined depending on the purpose of use (prophylactic, health, or therapeutic treatment). In general, when a food or a beverage is manufactured, the food composition of the present disclosure may be added in an amount of 50 parts by weight or less, specifically 20 parts by weight or less based on the total weight of the food or the beverage. However, when taken for the purpose of health and hygiene, the food composition may be contained in an amount below the range. In addition, since there is no safety problem, the active ingredients may be used in an amount above the range.

The food composition of the present disclosure may be used as, for example, a health beverage composition, and in this case, the health beverage composition may further contain various flavors or natural carbohydrates, as in common beverages. The natural carbohydrates may include monosaccharides such as glucose and fructose; disaccharides such as maltose and sucrose; polysaccharides such as dextrin and cyclodextrin; and sugar alcohols such as xylitol, sorbitol and erythritol. The sweeteners may be natural sweeteners such as thaumatin or a stevia extract; or synthetic sweeteners such as saccharin or aspartame. The natural carbohydrate may be generally used in an amount of from about 0.01 g to about 0.04 g, and specifically, from about 0.02 g to about 0.03 g based on 100 mL of the health beverage composition of the present disclosure.

In addition, the health beverage composition may contain various nutrients, vitamins, minerals, flavors, colorants, pectic acid and salts thereof, alginic acid and salts thereof, organic acid, protective colloidal thickeners, pH regulators, stabilizers, antiseptics, glycerin, alcohols, or carbonating agents. Moreover, the health beverage composition may contain fruit flesh used to prepare natural fruit juices, fruit juice beverages or vegetable beverages. These ingredients may be used individually or in combination. A proportion of the additives is not critical but is generally selected from 0.01 part by weight to 0.1 part by weight per 100 parts by weight of the health beverage composition of the present disclosure.

The food composition of the present disclosure may contain the mixed strains of Limosilactobacillus reuteri LM1071 and Lactobacillus gasseri LM1065 of the present disclosure in a variety of % by weight as long as it can exhibit the effect of improving protein proteolysis and lactose intolerance. Specifically, the mixed strains of Limosilactobacillus reuteri LM1071 and Lactobacillus gasseri LM1065 of the present disclosure may be contained in an amount of 0.00001% by weight to 100% by weight or 0.01% by weight to 80% by weight based on the total weight of the food composition but is not limited thereto.

A third aspect of the present disclosure provides a health functional food composition comprising the mixed strains of the first aspect or fragments, culture, and extracts of the strain as active ingredients and capable of improving whey protein proteolysis and lactose intolerance. The features described above in respect of the first and second aspects of the present disclosure may equally apply to health functional food composition according to the third aspect of the present disclosure.

In an embodiment of the present disclosure, the health functional food composition may contain the mixed strains of the present disclosure or fragments, a culture and extracts of the strain as active ingredients but is not limited thereto.

Through the whole document, the term “health functional food” refers to foods prepared and processed using raw materials or ingredients having useful functions to the human body in accordance with the Health Functional Food Act, No. 6727, and the “functionality” refers to adjusting nutrients on a structure and a function of the human body or obtaining a useful effect for health such as a physiological action.

The health functional food refers to a food having effects of actively maintaining or promoting health conditions, as compared with general foods, and a health supplement food refers to a food for supplementing health. If necessary, the health functional food, health food and health supplement food may be interchangeably used with each other. Specifically, the health functional food is a food prepared by adding the mixed strains of Limosilactobacillus reuteri LM1071 and Lactobacillus gasseri LM1065 of the present disclosure to food materials such as beverages, teas, spices, gums, confectionery, etc., or prepared in a capsule, a powder, or a suspension form. The health functional food means that it has a specific effect on health when consumed, but unlike general drugs, the health functional food is free from side effects that may occur when a drug is taken for a long time since the food is used as raw materials.

A fourth aspect of the present disclosure provides an infant formula composition comprising the mixed strains of the first aspect or fragments, culture and extracts of the strain as active ingredients and capable of improving protein proteolysis and lactose intolerance. The features described above in respect of the first to third aspects of the present disclosure may equally apply to the infant formula composition according to the fourth aspect of the present disclosure.

In an embodiment of the present disclosure, the infant formula composition may contain the mixed strains of the present disclosure or fragments, a culture and extracts of the strain as active ingredients but is not limited thereto.

A fifth aspect of the present disclosure provides a comprising administering to a mammalian subject effective amount of Limosilactobacillus reuteri LM1071 (Accession Number: KCCM12650P) and Lactobacillus gasseri LM1065 (Accession Number: KCCM13018P). The features described above in respect of the first to fourth aspects of the present disclosure may equally apply to the method according to the fifth aspect of the present disclosure. Administering effective amount of pharmaceutical composition of the mixed strains or the other could be an option for the treating method.

In an embodiment of the present disclosure, the pharmaceutical composition may contain the mixed strains of the present disclosure or fragments, a culture and extracts of the strain as active ingredients but is not limited thereto.

In an embodiment of the present disclosure, the pharmaceutical composition may be formulated and used as formulations for oral administration such as powders, granules, tablets, capsules, suspensions, emulsions, syrups or aerosol, external preparations, suppositories, or sterile injection solutions by conventional methods, respectively, but is not limited thereto.

In an embodiment of the present disclosure, the pharmaceutical composition may be formulated with generally used diluents or excipients such as fillers, bulking agents, binders, wetting agents, disintegrating agents or surfactants, but is not limited thereto.

In an embodiment of the present disclosure, solid formulations for oral administration may include tablets, pills, powders, granules or capsules, and these solid formulations may be prepared by mixing a component derived from the strain with at least one of excipients such as starch, calcium carbonate, sucrose, lactose, or gelatin. Except for the simple excipients, lubricants such as magnesium stearate or talc may be used, but the present disclosure is not limited thereto.

In an embodiment of the present disclosure, liquid formulations for oral administration may include suspensions, solutions for internal use, emulsions and syrups, and may contain various excipients such as wetting agents, sweeteners, aromatics and preservatives in addition to generally used simple diluents such as water and liquid paraffin but is not limited thereto.

In an embodiment of the present disclosure, formulations for parenteral administration may include sterilized aqueous solutions, water-insoluble excipients, suspensions, emulsions, lyophilized preparations, and suppositories, but is not limited thereto. For example, the water insoluble excipients or suspensions may contain propylene glycol, polyethylene glycol, vegetable oil such as olive oil, injectable ester such as ethylolate, and the like, but is not limited thereto. For example, the suppositories may contain witepsol, macrogol, tween 61, cacao butter, laurin butter, glycerol, gelatin, and the like, but is not limited thereto.

The pharmaceutical composition according to an embodiment of the present disclosure may be a drug composition or a quasi-drug composition.

Through the whole document, the term “quasi-drug” refers to an article having a milder action than drugs, among articles being used for the purpose of diagnosis, treatment, improvement, alleviation, handling or prevention of human or animal diseases. For example, according to the Pharmaceutical Affairs Law, the quasi-drugs are those, excluding articles used as drugs, including articles used for the purpose of treating or preventing human or animal diseases and articles which have a mild action on or have no direct influence on the human body.

The quasi-drug composition of the present disclosure may be manufactured in a formulation selected from the group consisting of body cleanser, sanitizer, detergent, kitchen cleanser, detergent for cleaning, toothpaste, mouthwash, wet wipe, cleanser, soap, hand soap, hair cleanser, hair softener, humidifying filler, mask, ointment, or filter filler, but is not limited thereto.

In an embodiment of the present disclosure, the pharmaceutical composition may be administered in a pharmaceutically effective amount. Through the whole document, the term “pharmaceutically effective amount” refers to an amount sufficient to treat or prevent diseases at a reasonable benefit/risk ratio applicable to any medical treatment or prevention. An effective dosage level may be determined depending on factors including severity of the disease, drug activity, a patient's age, body weight, health conditions, gender, sensitivity to the drug, administration time, administration route, and excretion rate of the composition of the present disclosure, duration of treatment, drugs blended with or co-administered with the composition of the present disclosure, and other factors known in the medical field. The pharmaceutical composition of the present disclosure may be administered individually or in combination with an ingredient known for treating intestinal diseases. It is important to administer an amount to obtain a maximum effect in a minimum amount without side effects by considering all the above-described factors.

In an embodiment of the present disclosure, an administration dose of the pharmaceutical composition may be determined by a person with ordinary skill in the art in view of purpose of use, severity of the disease, a patient's age, body weight, gender, medical history, or the kind of a material used as an active ingredient. For example, the pharmaceutical composition of the present disclosure may be administered at a dose of from about 0.1 ng/kg to about 1,000 mg/kg, and preferably, from about 1 ng/kg to about 100 mg/kg per adult, and the administration frequency of the composition of the present disclosure is not particularly limited, but the composition may be administered once a day or several times a day in divided doses. The administration dose or the administration frequency does not limit the scope of the present disclosure in any aspect.

The pharmaceutical composition of the present application may be administered via, but not particularly limited to, intraperitoneal administration, intravenous administration, intramuscular administration, subcutaneous administration, intradermal administration, transdermal patch administration, oral administration, intranasal administration, intrapulmonary administration, rectal administration, etc. depending on the purpose. However, when the pharmaceutical composition is administered via oral administration, it can be administered in an unformulated form, and since the mixed strains of Limosilactobacillus reuteri LM1071 and Lactobacillus gasseri LM1065 can be denatured or degraded by gastric acid, the composition for oral administration may be coated with an active drug, formulated to be protected from degradation in the stomach, or formulated in the form or an oral patch. Also, the composition may be administered by any device capable of delivering an active ingredient to a target cell.

Hereinafter, the present disclosure will be explained in more detail with reference to Examples. However, the following Examples are illustrative only for better understanding of the present disclosure but do not limit the present disclosure.

### EXAMPLES

### Example 1. Selection of Strain Having Excellent Whey Protein Proteolysis

Plate media were prepared from skimmed milk powder and agar to select strains having excellent whey protein proteolysis and then, whey protein proteolysis of each strain was identified. After 25 g of skimmed milk powder and 15 g of agar were dissolved and sterilized in 250 mL of distilled water and 500 mL of distilled water, respectively, the two solutions were mixed and 20 mL of the mixed solution was dispensed into each well, followed by cooling at room temperature to solidify the agar to be used. Pancreatin was used as a positive control group, which was prepared and used by dissolving pancreatin powder in a simulated intestinal buffer solution (0.5% NaCl, 0.03% CaCl₂, 0.06% KCl and 0.06% NaHCO₃) of pH 7.4 by a concentration of 0.01% (w/v; 100 μg/ml).

The strains used for strain selection were as shown in Table 1 and used to evaluate proteolysis after cultured three times in 12-hour intervals. The inoculum concentration for each culture was 0.1% v/v, and the strain culture temperature was adjusted to 37±3° C. The agar medium prepared from skimmed milk powder was treated with 4 μL of a culture fluid for each strain and placed at room temperature for 30 minutes in order for the culture fluid to sufficiently permeate into the medium, followed by culture in a 37° C. incubator for 24 hours. Then, the Image J program was used to check whether a clear zone is formed and to measure the area and transparency of the clear zone. The area of the clear zone was calculated by measuring the area of a part where a protein is degraded and becomes transparent and converted into mm², and the transparency was obtained by measuring a gray value of the clear zone and a gray value of the medium and calculating a protein degradation index according to the following formula. Every test was conducted three times, and the average of each result was obtained and compared.

$$Protein degradation index = ( A \times B ) / 100$$

A: Area of clear zone (mm²)

$$B : Transparency of clear zone ( \% ) = 100 - ( Gray value of clear zone / Gray value of culture medium ) \times 100$$

As a result, it was confirmed that all probiotics cannot degrade a whey protein and only a specific probiotic has excellent whey protein proteolysis. A strain L5 (Lactobacillus gasseri 5 LM1065 (KCCM13018P)) and a strain L7 (Limosilactobacillus reuteri LM1071 (KCCM12650P)) were selected as strains having excellent whey protein proteolysis from among probiotics derived from various origins in consideration of the protein degradation index (see Table 1 and Table 2).

TABLE 1
Strains used for comparison in terms of whey protein proteolysis.

| Abbreviation | Strain Name | Origin |
|---|---|---|
| B1 | Bifidobacterium animalis lactis | Infant feces |
| B2 | Bifidobacterium bifidum | Human breast milk |
| B3 | Bifidobacterium longum | Infant feces |
| L1 | Lacticaseibacillus rhamnosus | Cheese |
| L2 | Lacticaseibacillus rhamnosus | Infant feces |
| L3 | Lactiplantibacillus plantarum | Kimchi |
| L4 | Lactobacillus acidophilus | Adult feces |
| L5 | Lactobacillus gasseri | Human breast milk |
| L6 | Limosilactobacillus fermentum | Fermented dough |
| L7 | Limosilactobacillus reuteri | Human breast milk |
| S1 | Streptococcus thermophilus | Dairy product |

TABLE 2
Comparison of probiotics in terms of protein degradation index

| | | | Protein |
|---|---|---|---|
| | Area of | Transparency | Degradation |
| Classification | Clear Zone | of Clear Zone | Index |
| Agar Culture Medium | — | — | — |
| Positive Control Group¹⁾ | 44.2 | 21.6 | 9.6 |
| B1 | — | — | — |
| B2 | — | — | — |
| B3 | 25.1 | 6.2 | 1.6 |
| L1 | — | — | — |
| L2 | 34.2 | 11.6 | 4.0 |
| L3 | 34.1 | 10.7 | 3.6 |
| L4 | 73.6 | 7.7 | 3.5 |
| L5 | 44.2 | 9.8 | 4.3 |
| L6 | 47.9 | 7.8 | 3.8 |
| L7 | 38.5 | 21.1 | 8.1 |
| S1 | — | — | — |

¹⁾0.01% pancreatin

### Example 2. Change in Protein Proteolysis Depending on Mixing Ratio of Limosilactobacillus reuteri and Lactobacillus gasseri

Media were prepared from skimmed milk powder and agar in the same way as in Example 1 to evaluate protein proteolysis depending on a mixing ratio of Limosilactobacillus reuteri and Lactobacillus gasseri selected as probiotics having excellent protein proteolysis in Example 1. Then, whey protein proteolysis was identified by a plate culture method. A bromelain solution dissolved at 5% (w/v) in an acetate buffer of pH 4.5 was used as a positive control group.

The two strains selected as having excellent protein proteolysis in Example 1 were cultured three times in 12-hour intervals in the same way as in Example 1 and then used for protein proteolysis evaluation. The two strains were mixed at nine combinations of ratios as shown in Table 3 for comparison in terms of protein proteolysis. The strains were mixed at each ratio, and 10 μL of the mixture was inoculated into a skimmed milk powder medium and placed at room temperature for 30 minutes in order for a culture fluid to sufficiently permeate into the medium, followed by culture in a 37° C. incubator for 48 hours. The area and transparency of a clear zone were obtained, and a protein degradation index was calculated in the same way as in Example 1. A Predicted value of synergistic effect was calculated according to Colby's formula and compared with an actual Measurement.

$$Predicted value of synergistic effect = ( A + B ) - ( A \times B / 100 )$$

A: Measurement of protein proteolysis by Limosilactobacillus reuteri
B: Measurement of protein proteolysis by Lactobacillus gasseri

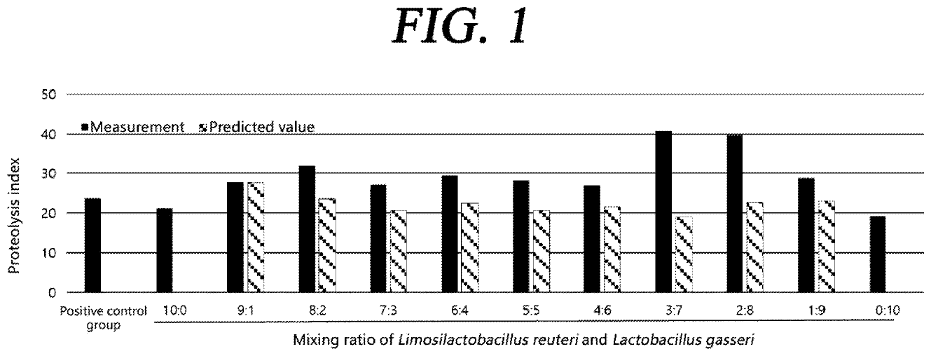

As a result of measuring a protein degradation index of the two selected strains at a single mixing ratio or at nine combinations of mixing ratios, all the mixed strains have higher protein proteolysis than bromelain, which is a positive control group, regardless a mixing ratio. Also, the protein degradation indexes measured at all the mixing ratios were higher than the Predicted values of synergistic effect, which confirms the synergistic effect caused by mixing of Limosilactobacillus reuteri and Lactobacillus gasseri. However, the proteolysis significantly varies depending on a mixing ratio of the strains, and in particular, the mixed strains of Limosilactobacillus reuteri and Lactobacillus gasseri showed the highest whey protein proteolysis at a mixing ratio of 3:7 (see FIG. 1 and Table 3).

TABLE 3
Change in protein proteolysis depending on mixing ratio of
Limosilactobacillus reuteri and Lactobacillus gasseri

| | | Measurement | | | Predicted value | | |
|---|---|---|---|---|---|---|---|
| | | | Trans- | Protein | | Trans- | Protein |
| | | Area | parency | degradation | Area | parency | degradation |
| | | (mm²) | (%) | index | (mm²) | (%) | index |
| 5% bromelain | | 81.5 | 29.5 | 23.7 | — | — | — |
| A¹⁾ | B²⁾ | | | | | | |
| 90% | 10% | 129.9 | 21.4 | 27.8 | 110.1 | 32.7 | 27.6 |
| 80% | 20% | 164.6 | 20.6 | 31.8 | 99.0 | 31.0 | 23.5 |
| 70% | 30% | 196.6 | 14.4 | 27.0 | 93.8 | 27.7 | 20.5 |
| 60% | 40% | 170.5 | 17.2 | 29.4 | 91.9 | 30.2 | 22.3 |
| 50% | 50% | 140.4 | 20.0 | 28.2 | 92.3 | 27.5 | 20.5 |
| 40% | 60% | 144.7 | 18.5 | 26.9 | 93.0 | 28.7 | 21.5 |
| 30% | 70% | 207.6 | 19.6 | 40.7 | 91.0 | 27.4 | 18.9 |
| 20% | 80% | 187.0 | 21.2 | 39.6 | 94.9 | 30.4 | 22.7 |
| 10% | 90% | 131.6 | 21.8 | 28.7 | 95.8 | 30.0 | 22.9 |
| — | 100% | 99.8 | 19.2 | 19.2 | — | — | — |
| — | 90% | 92.0 | 18.8 | 17.4 | — | — | — |
| — | 80% | 87.7 | 16.5 | 14.4 | — | — | — |
| — | 70% | 74.2 | 12.5 | 9.1 | — | — | — |
| — | 60% | 74.8 | 12.4 | 9.3 | — | — | — |
| — | 50% | 66.8 | 12.1 | 8.1 | — | — | — |
| — | 40% | 57.8 | 13.7 | 8.0 | — | — | — |
| — | 30% | 44.0 | 11.6 | 5.1 | — | — | — |
| — | 20% | 35.3 | 13.0 | 4.5 | — | — | — |
| — | 10% | 29.4 | 14.2 | 4.1 | — | — | — |
| 100% | — | 114.8 | 18.6 | 21.2 | — | — | — |
| 90% | — | 114.3 | 21.6 | 24.4 | — | — | — |
| 80% | — | 98.4 | 20.7 | 19.9 | — | — | — |
| 70% | — | 89.0 | 18.1 | 16.2 | — | — | — |
| 60% | — | 80.9 | 19.1 | 15.5 | — | — | — |
| 50% | — | 76.7 | 17.6 | 13.5 | — | — | — |
| 40% | — | 72.4 | 18.7 | 13.5 | — | — | — |
| 30% | — | 65.2 | 16.9 | 10.8 | — | — | — |
| 20% | — | 58.5 | 16.6 | 9.7 | — | — | — |
| 10% | — | 47.5 | 13.9 | 6.6 | — | — | — |

¹⁾Limosilactobacillus reuteri
²⁾Lactobacillus gasseri

### Example 3. Change in Branched Chain Amino Acid Production Potential after WPC Degradation Depending on Mixing Ratio of Limosilactobacillus reuteri and Lactobacillus gasseri

After a mixed composition of Limosilactobacillus reuteri and Lactobacillus gasseri was cultured with WPC, changes in content of branched chain amino acids (valine, isoleucine, and leucine) produced by degradation of WPC were analyzed using high-performance liquid chromatography with photodiode array detection.

A 2.5% (w/v) WPC medium was prepared by dissolving 12.5 g of WPC powder in 500 mL of sterile water, dispensing 30 mL of the solution into a 50 mL conical tube and performing sterilization at 65° C. for 30 minutes. Strains were cultured three times in the same way as in Example 1 and then collected to be diluted to 8 log CFU/ml in a phosphate buffered saline, followed by inoculation of an individual strain or mixed strains at a concentration of 0.1% v/v in the WPC medium. Thereafter, culture was performed in a 37±3° C. incubator for 72 hours, followed by centrifugation (4000 rpm, 15 minutes) of the culture fluid. The supernatant was used as an analyte.

After 5 mL of the analyte was put into a test tube and concentrated at 110° C. under a nitrogen environment and finely ground, the resultant product was dissolved in 1 ml of a 0.1 N aqueous hydrochloric acid solution, followed by homogenization through vortexing. Then, free amino acids were extracted from the concentration sample in an ultrasonic water bath for 15 minutes. The free amino acid extraction fluid was centrifuged to filter the supernatant through a filter. The free amino acids were analyzed using high-performance liquid chromatography with photodiode array detection. The analysis was performed with a gradient elution by using a 0.1% aqueous formate solution and a 0.1% formate acetonitrile solution as a mobile phase, and an Agilent Zorbax Eclipse AAA column (4.6 mm ID*150 mm, 5 μm) was used for analysis.

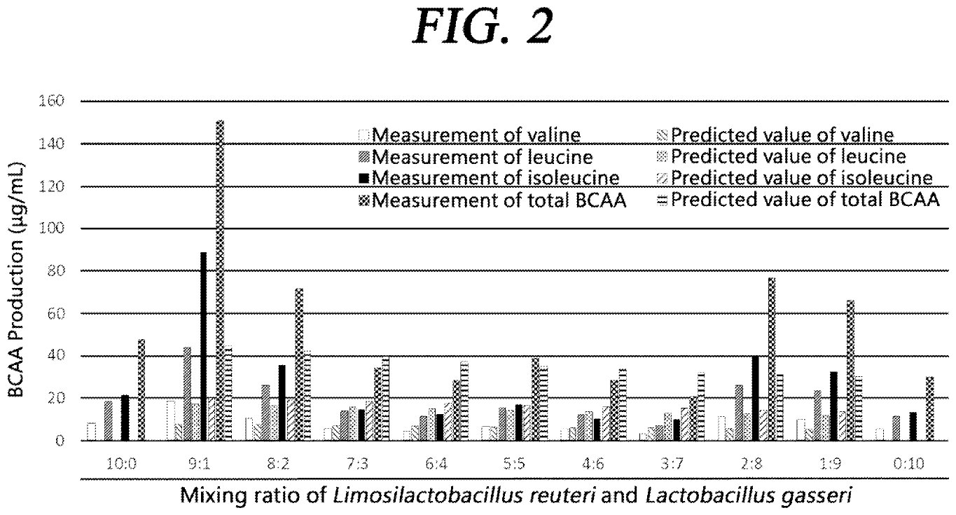

As a result, branched chain amino acids were detected from WPC in a minute amount lower than the limit of quantitation, whereas branched chain amino acids were detected in a large amount from a culture fluid where WPC and probiotics were co-cultured. It was confirmed that both Limosilactobacillus reuteri and Lactobacillus gasseri degraded WPC to branched chain amino acids, and when the strains were mixed, the amount of branched chain amino acids further increased. The content of branched chain amino acids greatly varies depending on a mixing ratio of the strains. As confirmed in Example 2, Limosilactobacillus reuteri and Lactobacillus gasseri in the skimmed milk powder showed the highest protein proteolysis at a mixing ratio of 3:7 and the highest potential of degrading WPC to branched chain amino acids at a mixing ratio of 9:1 (see FIG. 2 and Table 4).

TABLE 4
Difference in branched chain amino acid production potential depending on
mixing ratio of Limosilactobacillus reuteri and Lactobacillus gasseri

| | | Measurement(μg/mL) | | | | Predicted value(μg/mL) | | | |
|---|---|---|---|---|---|---|---|---|---|
| | | | | | Sum of | | | | Sum of |
| | | | | | branched | | | | branched |
| | | | | | chain | | | | chain |
| | | | | | amino | | | | amino |
| A¹⁾ | B²⁾ | Valine | Isoleucine | Leucine | acids | Valine | Isoleucine | Leucine | acids |
| 90% | 10% | 18.6 | 43.6 | 88.8 | 150.9 | 7.9 | 17.3 | 20.2 | 44.6 |
| 80% | 20% | 10.2 | 25.9 | 35.4 | 71.4 | 7.6 | 16.5 | 19.2 | 41.9 |
| 70% | 30% | 6.0 | 13.9 | 14.6 | 34.5 | 7.3 | 15.7 | 18.3 | 39.4 |
| 60% | 40% | 4.6 | 11.2 | 12.3 | 28.1 | 7.0 | 15.0 | 17.4 | 37.2 |
| 50% | 50% | 6.6 | 15.5 | 16.8 | 38.9 | 6.7 | 14.3 | 16.5 | 35.2 |
| 40% | 60% | 5.6 | 12.2 | 10.3 | 28.1 | 6.4 | 13.6 | 15.7 | 33.6 |
| 30% | 70% | 3.3 | 7.3 | 10.0 | 20.6 | 6.2 | 13.0 | 15.0 | 32.3 |
| 20% | 80% | 11 | 26.1 | 39.2 | 76.3 | 5.9 | 12.4 | 14.3 | 31.2 |
| 10% | 90% | 9.8 | 23.6 | 32.7 | 66.1 | 5.7 | 11.9 | 13.7 | 30.4 |
| 100% | — | 8.2 | 18.2 | 21.3 | 47.6 | — | — | — | — |
| 90% | — | 7.4 | 16.4 | 19.2 | 42.9 | — | — | — | — |
| 80% | — | 6.6 | 14.6 | 17 | 38.2 | — | — | — | — |
| 70% | — | 5.7 | 12.7 | 14.9 | 33.4 | — | — | — | — |
| 60% | — | 4.9 | 10.9 | 12.8 | 28.6 | — | — | — | — |
| 50% | — | 4.1 | 9.1 | 10.7 | 23.9 | — | — | — | — |
| 40% | — | 3.3 | 7.3 | 8.5 | 19.1 | — | — | — | — |
| 30% | — | 2.5 | 5.5 | 6.4 | 14.3 | — | — | — | — |
| 20% | — | 1.6 | 3.6 | 4.3 | 9.5 | — | — | — | — |
| 10% | — | 0.8 | 1.8 | 2.1 | 4.8 | — | — | — | — |
| — | 100% | 5.4 | 11.4 | 13.1 | 29.9 | — | — | — | — |
| — | 90% | 4.9 | 10.3 | 11.8 | 27.0 | — | — | — | — |
| — | 80% | 4.3 | 9.1 | 10.5 | 23.9 | — | — | — | — |
| — | 70% | 3.8 | 8.0 | 9.2 | 21.0 | — | — | — | — |
| — | 60% | 3.1 | 6.8 | 7.9 | 17.9 | — | — | — | — |
| — | 50% | 2.6 | 5.7 | 6.6 | 15.0 | — | — | — | — |
| — | 40% | 2.2 | 4.6 | 5.2 | 12.0 | — | — | — | — |
| — | 30% | 1.6 | 3.4 | 3.9 | 9.0 | — | — | — | — |
| — | 20% | 1.1 | 2.3 | 2.6 | 6.0 | — | — | — | — |
| — | 10% | 0.5 | 1.1 | 1.3 | 3.0 | — | — | — | — |

¹⁾Limosilactobacillus reuteri
²⁾Lactobacillus gasseri

### Example 4. Change in Arginine and Phenylalanine Production Potential after WPC Degradation Depending on Mixing Ratio of Limosilactobacillus reuteri and Lactobacillus gasseri

After a mixed composition of Limosilactobacillus reuteri and Lactobacillus gasseri was cultured with WPC, changes in content of arginine and phenylalanine produced by degradation of WPC were analyzed using high-performance liquid chromatography with photodiode array detection in the same way as in Example 3.

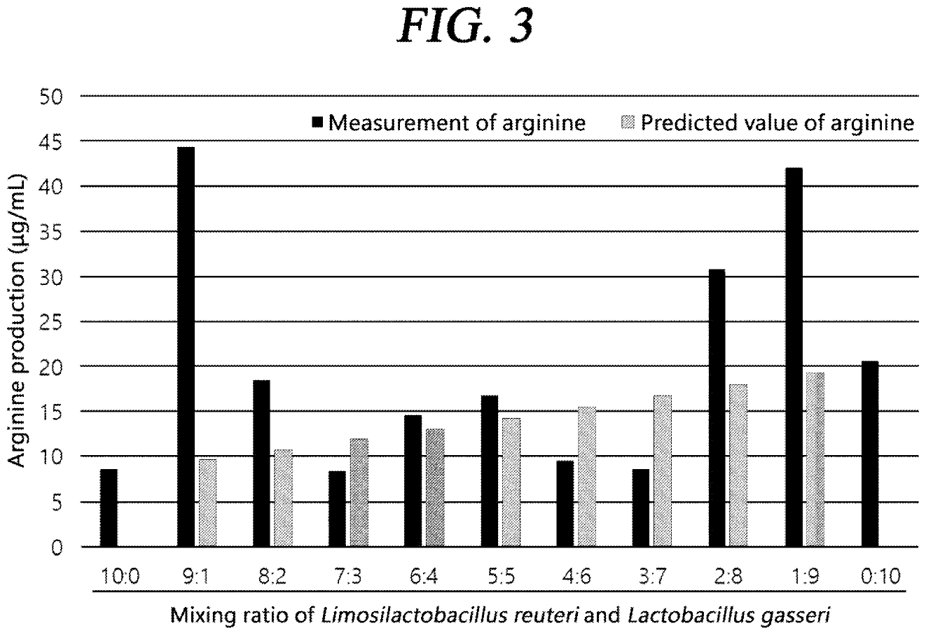

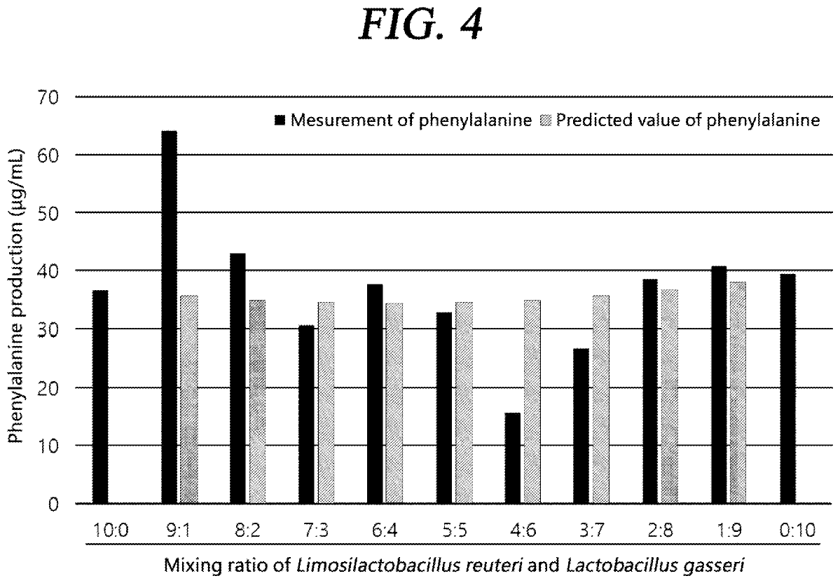

As a result, arginine was detected from a culture fluid prepared from WPC in a minute amount lower than the limit of quantitation and phenylalanine was also detected in a minute amount higher than the limit of quantitation. Meanwhile, arginine and phenylalanine were detected in a large amount from a culture fluid where WPC and probiotics were co-cultured. It was confirmed that both Limosilactobacillus reuteri and Lactobacillus gasseri degraded WPC to arginine and phenylalanine, and when the strains were mixed, the amounts of arginine and phenylalanine further increased. The content of arginine greatly varies depending on a mixing ratio of the strains. As confirmed in Example 3, Limosilactobacillus reuteri and Lactobacillus gasseri in WPC showed the highest branched chain amino acid production potential at a mixing ratio of 9:1 and the highest potential of producing arginine and phenylalanine at a mixing ratio of 9:1 (see FIG. 3, FIG. 4, Table 5 and Table 6).

TABLE 5
Difference in arginine production potential depending on mixing ratio
of Limosilactobacillus reuteri and Lactobacillus gasseri

| | | Measurement | Predicted |
|---|---|---|---|
| | | (μg/mL) | value(μg/mL) |
| A¹⁾ | B²⁾ | Arginine | Arginine |
| 90% | 10% | 44.3 | 9.6 |
| 80% | 20% | 18.4 | 10.7 |
| 70% | 30% | 8.3 | 11.8 |
| 60% | 40% | 14.5 | 13.0 |
| 50% | 50% | 16.8 | 14.2 |
| 40% | 60% | 9.5 | 15.4 |
| 30% | 70% | 8.6 | 16.6 |
| 20% | 80% | 30.8 | 17.9 |
| 10% | 90% | 42.0 | 19.2 |
| 100% | — | 8.6 | — |
| 90% | — | 7.7 | — |
| 80% | — | 6.9 | — |
| 70% | — | 6.0 | — |
| 60% | — | 5.2 | — |
| 50% | — | 4.3 | — |
| 40% | — | 3.4 | — |
| 30% | — | 2.6 | — |
| 20% | — | 1.7 | — |
| 10% | — | 0.9 | — |
| — | 100% | 20.6 | — |
| — | 90% | 18.5 | — |
| — | 80% | 16.5 | — |
| — | 70% | 14.4 | — |
| — | 60% | 12.4 | — |
| — | 50% | 10.3 | — |
| — | 40% | 8.2 | — |
| — | 30% | 6.2 | — |
| — | 20% | 4.1 | — |
| — | 10% | 2.1 | — |

¹⁾Limosilactobacillus reuteri
²⁾Lactobacillus gasseri

TABLE 6
Difference in phenylalanine production potential depending on mixing
ratio of Limosilactobacillus reuteri and Lactobacillus gasseri

| | | Measurement | Predicted value |
|---|---|---|---|
| | | (μg/mL) | (μg/mL) |
| A¹⁾ | B²⁾ | Phenylalanine | Phenylalanine |
| 90% | 10% | 64.1 | 35.6 |
| 80% | 20% | 43.0 | 34.9 |
| 70% | 30% | 30.5 | 34.4 |
| 60% | 40% | 37.6 | 34.3 |
| 50% | 50% | 32.8 | 34.4 |
| 40% | 60% | 15.7 | 34.9 |
| 30% | 70% | 26.8 | 35.6 |
| 20% | 80% | 38.6 | 36.6 |
| 10% | 90% | 40.7 | 37.9 |
| 100% | — | 36.6 | — |
| 90% | — | 32.9 | — |
| 80% | — | 29.3 | — |
| 70% | — | 25.6 | — |
| 60% | — | 22.0 | — |
| 50% | — | 18.3 | — |
| 40% | — | 14.6 | — |
| 30% | — | 11.0 | — |
| 20% | — | 7.3 | — |
| 10% | — | 3.7 | — |
| — | 100% | 39.5 | — |
| — | 90% | 35.5 | — |
| — | 80% | 31.6 | — |
| — | 70% | 27.6 | — |
| — | 60% | 23.7 | — |
| — | 50% | 19.7 | — |
| — | 40% | 15.8 | — |
| — | 30% | 11.8 | — |
| — | 20% | 7.9 | — |
| — | 10% | 3.9 | — |

¹⁾Limosilactobacillus reuteri
²⁾Lactobacillus gasseri

### Example 5. Change in Enzyme Activity Depending on Mixing Ratio of Limosilactobacillus reuteri and Lactobacillus gasseri

The API ZYM kit was used to evaluate enzyme activity of Limosilactobacillus reuteri and Lactobacillus gasseri and changes in enzyme activity depending on a mixing ratio of Limosilactobacillus reuteri and Lactobacillus gasseri. Microbial cells of Limosilactobacillus reuteri or Lactobacillus gasseri cultured three times in the same way as in Example 1 were collected, and a phosphate buffered saline was used to prepare a sample with a MacFarland turbidity of 5 according to an API ZM Kit usage (9.18 log CFU/MI). Then, 65 μl of the sample was placed in each strip in the kit and then cultured in a 37° C. incubator for 2 hours, followed by a colorimetric reaction through treatment with ZYM reagent to identify activity of each enzyme. Changes in the colorimetric reaction are represented from 0 to 5. A negative reaction is reported as “−”, a reaction with the maximum yield (40 nanomoles) is reported as “+++++”, and median value of 30, 20, 10 and 5 nanomoles are reported as “++++, +++, ++ and +”, respectively.

Enzyme activity evaluation was conducted onto individual strains, Limosilactobacillus reuteri and Lactobacillus gasseri, a 3:7 mixed composition having the highest whey protein proteolysis and a 9:1 mixed composition having the highest potential of producing branched chain amino acids and arginine from WPC by using the API ZYM Kit. As a result, it was confirmed that some lipid metabolic enzymes, protein metabolic enzymes and sugar metabolic enzymes differ in enzyme activity between the two individual strains, and mixing of the strains causes a difference in enzyme activity (see Table 7).

As a result of enzyme activity evaluation, it was confirmed that both the strains are safe strains because they do not show activity of β-glucuronidase, an enzyme that produces amines and toxic substances during metabolism and is involved in causing colorectal cancer resulting from gastrointestinal mucosal damage and various diseases.

Activity of β-galactosidase, which is a lactase, was observed only in Limosilactobacillus reuteri but not in Lactobacillus gasseri. However, when the two strains were mixed, the mixed strains showed the highest enzyme activity of β-galactosidase regardless a mixing ratio. Thus, it was confirmed that when mixing of the two strains enables improvement of protein proteolysis, promotion of branched chain protein production through degradation of WPC, and digestion of lactose and thus can overcome the problems caused by high protein intake.

Also, it is known that when a deficiency of intestinal α-galactosidase induces non-digestible oligosaccharides, such as raffinose and stachyose to reach the large intestine without being digested and serve as a fermentable substrate to over-produce methane, carbon dioxide and hydrogen gases caused by anaerobic bacteria in the large intestine and thus causes abdominal distension and increases nitrogen emissions in the feces which smell foul. Lactobacillus gasseri did not show activity of α-galactosidase, but when it was mixed with Limosilactobacillus reuteri, activity of α-galactosidase was maintained, which confirmed that it is possible to solve abdominal distension and foul-smelling stools.

Meanwhile, it is known that mannosidase and flucosidase inhibit the formation of carbohydrate and have negative effects on energy intake, but both the strains did not show activity of flucosidase. However, it was confirmed that Lactobacillus gasseri shows activity of α-mannosidase and thus has negative effects on energy metabolism, but when it was mixed with Limosilactobacillus reuteri, activity of α-mannosidase was not shown. Also, leucine arylamidase and valine arylamidase that have effects on protein synthesis did not greatly differ in activity from when they were mixed. Therefore, it was confirmed that the mixed composition of the present disclosure does not have negative effects on energy metabolism while having positive effects on in vivo protein synthesis.

TABLE 7
Evaluation of enzyme activity caused by mixing of
Limosilactobacillus reuteri and Lactobacillus gasseri

| | | Mixing ratio of Limosilactobacillus | | | |
|---|---|---|---|---|---|
| | | reuteri:Lactobacillus gasseri | | | |
| | Enzyme name | 10:0 | 9:1 | 3:7 | 0:10 |
| 1 | Alkaline phosphatase | − | + | + | + |
| 2 | Esterase (C4) | +++ | ++ | ++ | + |
| 3 | Esterase Lipase (C8) | ++ | ++ | ++ | + |
| 4 | Lipase (C14) | − | + | + | + |
| 5 | Leucine arylamidase | +++++ | ++++ | ++++ | +++++ |
| 6 | Valine arylamidase | + | +++ | +++ | +++++ |
| 7 | Crystine arylamidase | + | +++ | +++ | +++++ |
| 8 | Trypsin | − | − | − | − |
| 9 | α-chymotrypsin | − | − | − | − |
| 10 | Acid phosphatase | +++++ | ++++ | ++++ | ++++ |
| 11 | Naphtol-AS-BI- | +++++ | +++++ | +++++ | ++++ |
| | Phosphohydrolase | | | | |
| 12 | α-galactosidase | +++++ | +++++ | +++++ | − |
| 13 | β-galactosidase | +++++ | +++++ | +++++ | − |
| 14 | β-glucuronidase | − | − | − | − |
| 15 | α-glucosidase | − | ++ | ++ | +++ |
| 16 | β-glucosidase | − | +++ | +++ | +++++ |
| 17 | N-acetyl-β-glucosaminidase | − | ++ | ++ | +++ |
| 18 | α-mannosidase | − | − | − | +++ |
| 19 | α-flucosidase | − | − | − | − |

### Example 6. Acid Resistance, Bile-Acid Resistance, Self-Aggregation, and Intestinal Adhesion of Mixed Strain

To predict viability of Limosilactobacillus reuteri and Lactobacillus gasseri in the digestive tract, acid resistance, Bile-acid resistance, self-aggregation and intestinal adhesion were evaluated, and changes in acid resistance, Bile-acid resistance, self-aggregation and intestinal adhesion caused by a mixing ratio of Limosilactobacillus reuteri and Lactobacillus gasseri were evaluated. Lacticaseibacillus rhamnosus GG (ATCC 53103) was treated together as a comparative strain to compare the result in evaluation.

To evaluate the acid resistance, a medium for acid resistance evaluation was prepared by using 0.1 N hydrochloric acid to dissolve pepsin in MRS broth of pH 2.5 to 0.3% (w/v) and dispensing 9 mL of the solution into a 15 ml conical tube. After 1 mL of Limosilactobacillus reuteri and Lactobacillus gasseri, which were cultured three times in the same way as in Example 1, or a mixture prepared at each mixing ratio was dispensed into the medium for acid resistance evaluation, followed by culture in a 37° C. incubator for 2 hours. The viable cell counts before and after culture were measured by a plate culture method to evaluate the acid resistance according to the following formula.

$$Acid resistance ( \% ) = ( A / B ) \times 100 \%$$

A: Initial microbial count (log CFU/mL)
B: Microbial count after 2 hours (log CFU/mL)

To evaluate the Bile-acid resistance, a medium for Bile-acid resistance evaluation was prepared by dissolving bile powder in MRS broth to 0.3% (w/v) and dispensing 9 mL of the solution into a 15 ml conical tube. After 1 mL of Limosilactobacillus reuteri and Lactobacillus gasseri, which were cultured three times in the same way as in Example 1, or a mixture prepared at each mixing ratio was dispensed into the medium for Bile-acid resistance evaluation, followed by culture in a 37° C. incubator for 24 hours. The viable cell counts before and after culture were measured to evaluate the Bile-acid resistance according to the following formula.

$$Bile - acid resistance ( \% ) = ( A / B ) \times 100 \%$$

A: Initial microbial count (log CFU/mL)
B: Microbial count after 24 hours (log CFU/mL)

To evaluate the self-aggregation, Limosilactobacillus reuteri or Lactobacillus gasseri, which was cultured three times in the same way as in Example 1, was centrifuged (10,000 rpm, 10 minutes) to collect the precipitated microbial cells, followed by washing three times with a phosphate buffered saline. Then, each microbial cell was diluted in a phosphate buffered saline so that the absorbance at 600 nm was regulated to 0.5±0.05. Compositions with different mixing ratios were prepared by mixing microbial cell samples of individual strains at respective ratios, diluting the mixed microbial cells in a phosphate buffered saline, and regulating the absorbance to 0.5±0.05 at 600 nm. After 5 mL of each prepared sample was dispensed into a 15 ml conical tube and cultured in a 37° C. water bath for 24 hours, the absorbances before and after culture were measured to evaluate the self-aggregation according to the following formula.

$$Self - aggregation ( \% ) = ( 1 - B / A ) \times 100 \%$$

A: Initial absorbance
B: Absorbance after 24 hours

To evaluate the intestinal adhesion, human intestinal epithelial cells (HT-29 cells) were used. The intestinal epithelial cells were cultured in a 75 mL T-flask to a concentration of 1.5×10⁶ cells/12 mL. After 50% of the cells were concentrated in the flask, the cells were dispensed into a 24 plate well to 1.0×10⁵ cells/well/mL. Then, the medium was replaced with an RPMI medium comprising 10% fetal bovine serum in 2-day intervals, and whether a monolayer is formed was checked.

To treat the HT-29 cell monolayer with the strains, Limosilactobacillus reuteri and Lactobacillus gasseri were cultured three times in the same way as in Example 1 and centrifuged at 10,000 rpm for 10 minutes to collect the precipitated microbial cells. The collected microbial cells were washed three times with a phosphate buffered saline, followed by regulation of the final microbial count to 8 log CFU/mL with an RPMI medium not comprising fetal bovine serum.

A culture fluid of the intestinal epithelial cells including the formed monolayer was removed and the intestinal epithelial cells were washed three times with a phosphate buffered saline (DPBS) used as a buffer solution for cell culture and then treated with 1 mL of each prepared strain or mixed composition of strains, followed by culture in a 37° C. incubator in which the atmosphere composition was adjusted to 5% carbon dioxide for 2 hours. Then, the culture fluid was removed, followed by washing twice with a phosphate buffered saline (DPBS). Thereafter, cells and microbial cells were collected by treatment with 1 mL of a solution in which Triton-X was dissolved in purified water to 0.1% (v/v). The intestinal adhesion activity was evaluated by measuring the viable cell counts before and after culture by a plate culture method and calculating the intestinal adhesion according to the following formula.

$$Intestinal adhesion = ( A / B ) \times 100 \%$$

A: Initial microbial count (log CFU/mL)
B: Microbial count after 2 hours (log CFU/mL)

As a result of research, it was confirmed that Limosilactobacillus reuteri and Lactobacillus gasseri were better in terms of the acid resistance, self-aggregation, and intestinal adhesion than the comparative strain. Also, when the strains were mixed at different mixing ratios, the acid resistance, self-aggregation and intestinal adhesion tended to vary depending on a mixing ratio, but the strains showed higher activity at all the mixing ratios than the comparative strain (see Table 8).

TABLE 8
Difference in acid resistance, Bile-acid resistance, self-aggregation
and intestinal adhesion depending on mixing ratio of
Limosilactobacillus reuteri and Lactobacillus gasseri

| | | Acid | Bile-acid | Self- | Intestinal |
|---|---|---|---|---|---|
| | | resistance | resistance | aggregation | adhesion |
| | | (%) | (%) | (%) | (%) |
| Comparative | | 99.0 | 97.1 | 52.0 | 79.6 |
| strain | | | | | |
| A¹⁾ | B²⁾ | | | | |
| 100% | — | 99.4 | 96.9 | 67.3 | 87.0 |
| 90% | 10% | 98.8 | 89.9 | 65.2 | 87.4 |
| 80% | 20% | 99.7 | 89.6 | 64.3 | 88.7 |
| 70% | 30% | 99.8 | 89.3 | 61.1 | 86.8 |
| 60% | 40% | 100.0 | 89.6 | 58.2 | 88.2 |
| 50% | 50% | 100.6 | 87.3 | 60.8 | 87.5 |
| 40% | 60% | 100.7 | 84.8 | 55.1 | 87.2 |
| 30% | 70% | 101.6 | 83.3 | 57.7 | 83.9 |
| 20% | 80% | 99.9 | 83.5 | 57.2 | 84.1 |
| 10% | 90% | 99.9 | 77.4 | 56.9 | 82.9 |
| — | 100% | 98.7 | 52.2 | 50.4 | 82.9 |

¹⁾Limosilactobacillus reuteri
²⁾Lactobacillus gasseri

In sum, it was confirmed that the mixed strains of the present disclosure or the composition comprising a culture of the mixed strains has excellent probiotic characteristics such as intestinal viability and adhesion, degrades whey proteins, has the potential of producing valine, leucine and isoleucine, which are branched chain amino acids, arginine, and phenylalanine from whey protein concentrate, and shows activity of α-galactosidase, which is an enzyme involved in inhibiting the formation of intestinal gas, and β-galactosidase, which hydrolyzes lactose, and, thus, the mixed strains or mixed strains composition is effective in relieving the side effects associated with protein digestion and forming muscles in case of protein supplement intake.

The above description of the present disclosure is provided for the purpose of illustration, and it would be understood by a person with ordinary skill in the art that various changes and modifications may be made without changing technical conception and essential features of the present disclosure. Thus, the above-described examples are illustrative in all aspects and do not limit the present disclosure. For example, each component described to be of a single type can be implemented in a distributed manner. Likewise, components described to be distributed can be implemented in a combined manner.

1. A composition comprising mixed strains of Limosilactobacillus reuteri LM1071 deposited to Korean Culture Center of Microorganisms under the accession number KCCM12650P and Lactobacillus gasseri LM1065 deposited to Korean Culture Center of Microorganisms under the accession number KCCM13018P) and capable of improving whey protein proteolysis and lactose intolerance.

2. The composition of claim 1,
wherein the mixed strains have a mixing ratio of Limosilactobacillus reuteri LM1071 and Lactobacillus gasseri LM1065 of 2:8 to 4:6.

3. The composition of claim 1,
wherein the mixed strains have branched chain amino acid, arginine or phenylalanine production potential.

4. The composition of claim 3,
wherein the mixed strains have a mixing ratio of Limosilactobacillus reuteri LM1071 and Lactobacillus gasseri LM1065 of 9:1 to 6:4.

5. The composition of claim 1,
wherein the mixed strains have α-galactosidase activity and β-galactosidase activity.

6. A food composition comprising one or more of the mixed strains of claim 1 or a culture, fragments, and extracts of the strain as active ingredients.

7. A health functional food composition comprising one or more of the mixed strains of claim 1 or a culture, fragments, and extracts of the strain as active ingredients.

8. An infant formula composition comprising one or more of the mixed strains of claim 1 or a culture, fragments, and extracts of the strain as active ingredients.

9. A pharmaceutical composition for treating lactose intolerance or sarcopenia, containing one or more of the mixed strains of claim 1 or a culture, fragments, and extracts of the strain as active ingredients.
